(19) United States
(12) Plant Patent Application Publication
(10) Pub. No.: US 2003/0079264 P1
(43) Pub. Date: Apr. 24, 2003

(54) Strawberry plant named Madeira

(76) Inventors: Kristie L. Gilford, Dover, FL (US); Bruce D. Mowrey, Watsonville, CA (US)

Correspondence Address: PENNIE & EDMONDS LLP, 1155 Avenue of the Americas, New York, NY, 10036-2711, US

(21) Appl. No.: 09/968,558

(22) Filed: Oct. 1, 2001

(51) Int. Cl. A01H 5/00

(52) U.S. Cl. PLT/209

(57) ABSTRACT

This invention relates to a new and distinct variety of strawberry named ‘Madeira’. The variety is similar to the varieties ‘Marathon’ and ‘Biscayne’. The variety is distinguished from Marathon and Biscayne, in particular, by its flat habit, dense plant density, obtuse terminal leaflet shape, same size calyx in relation to fruit on the secondary fruit, and fruit with weak to medium acidity.

### BACKGROUND OF THE INVENTION

The new variety originated as a result of a controlled cross between the strawberry 7C171 and 21Y13 (both unpatented varieties) in an ongoing breeding program, and was discovered as a seedling in a controlled breeding plot in Hillsborough County, Florida in December, 1997. The original seedling was asexually propagated by stolons in a nursery in Shasta County, California. Propagules were transplanted to a controlled breeding plot in Hillsborough County, Florida where the variety was identified and selected for further evaluation. Madeira was subsequently asexually propagated and underwent further testing in Hillsborough County, Florida for five years. This propagation and testing has demonstrated that the combination of traits disclosed herein which characterize the new variety are fixed and retained true to type through successive generations of asexual reproduction.

### SUMMARY OF THE INVENTION

The present invention relates to a new and distinct variety of strawberry named ‘Madeira’. The variety is botanically identified as Fragaria×ananassa. The new variety is distinguished from other varieties by a number of characteristics as set forth in Tables 1-4.

### COMPARISON TO SIMILAR VARIETIES

The varieties which we believe to be similar to Madeira from those known to us are ‘Biscayne’ (U.S. patent application Ser. No. 09/396,214 filed Sep. 15, 1999) and ‘Marathon’ (U.S. patent application Ser. No. 09/396,213 filed Sep. 15, 1999). There are several characteristics of the new variety that are different from, or not possessed by Biscayne or Marathon. The base of the terminal leaflet shape is obtuse. The pose of the petiole hairs is downwards. The size of the calyx in relation to fruit on the secondary fruit is smaller.

### BRIEF DESCRIPTION OF THE DRAWINGS

The accompanying photographs show typical specimens of the new variety, including fruit, foliage and flowers, in color as nearly true as it is reasonably possible to make in color illustrations of these characteristics.

FIG. 1 shows the whole plant.

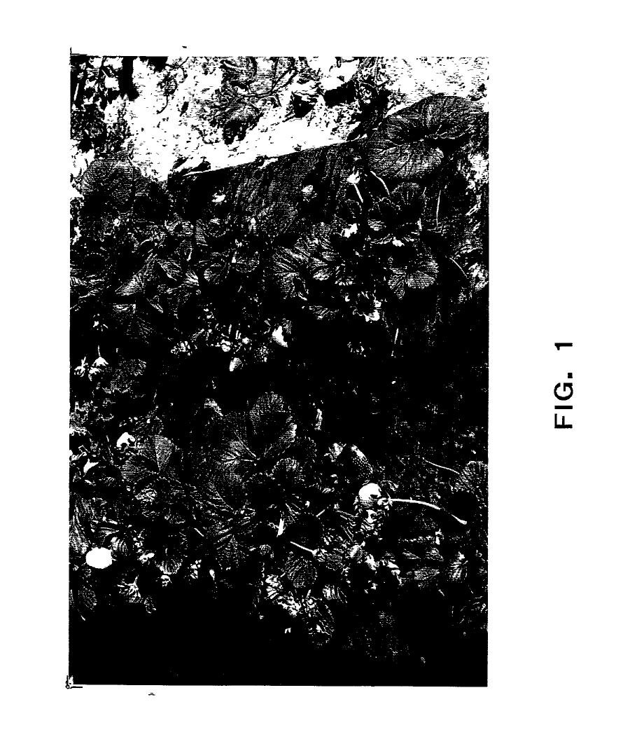

FIG. 2 shows the leaves of the plant.

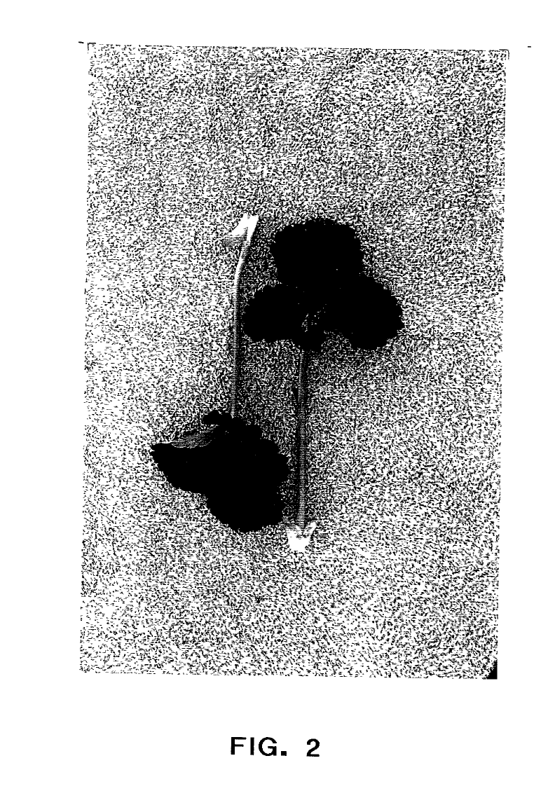

FIG. 3 shows the upper side and the under side of the flowers.

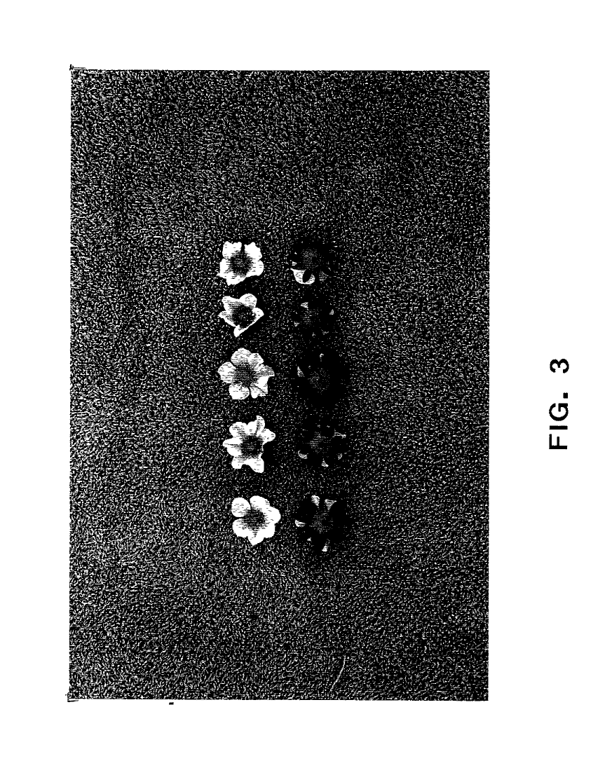

FIG. 4 shows a close-up of the fruit.

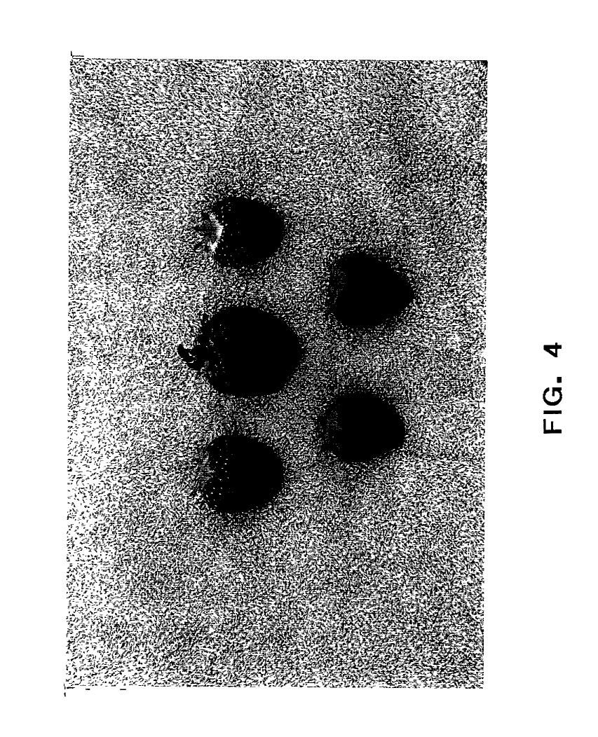

FIG. 5 shows the fruit in longitudinal cross-section.

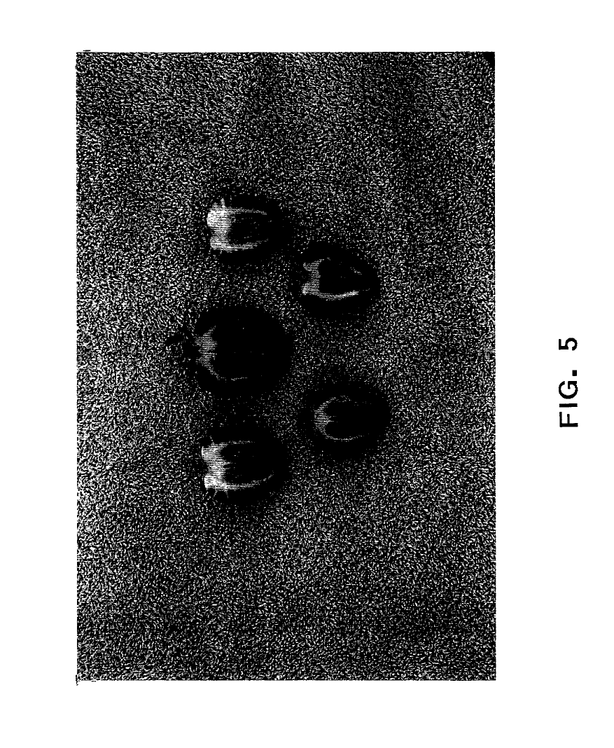

### DESCRIPTION OF THE NEW VARIETY

The following detailed description of the new variety is based upon observations taken of plants and fruit grown in Hillsborough, Fla., USA. Observations of Madeira, Marathon, and Biscayne were taken in side by side comparison in January, 2001. This description is in accordance with UPOV terminology. Color designations, color descriptions, and other phenotypical descriptions may deviate from the stated values and descriptions depending upon variation in environmental, seasonal, climatic and cultural conditions. Colors are described and the most similar color designations are provided from The Royal Horticultural Society (RHS) Colour Chart.

### Propagation

The new variety is principally propagated by way of stolons or by tissue culture. Although propagation by stolons or tissue culture is presently preferred, other known methods of propagating strawberry plants may be employed.

### Characteristics of the New Variety

Information on the new variety is presented in Tables 1, 2, 3 and 4. In the tables, the flowers described are secondary flowers except where indicated. The petal color of Madeira is white, 155C in the RHS Color Chart. The fruit described is the secondary fruit on one year old plants. Fruit and flower measurements are an average of both primary and secondary fruit and flowers.

Table 1 provides information on the plant and fruit characteristics of the new variety Madeira compared with characteristics of Marathon and Biscayne. Table 2 provides additional information of the plant and fruit characteristics of the new variety Madeira compared with characteristics of the varieties Marathon and Biscayne. Table 3 provides information of the new variety's reaction to pests and diseases, respectively, compared to the varieties Marathon and Biscayne. Table 4 provides isozyme characteristics of the new variety as compared to the varieties Marathon and Biscayne.

TABLE 1
DETAILED COMPARISON OF MADEIRA, BISCAYNE AND
MARATHON

| | Madeira | Biscayne | Marathon |
|---|---|---|---|
| Plant Characteristics | | | |
| Height of Plant (cm) | 17 | 13.7 | 14.3 |
| Spread of Plant (cm) | 28.2 | 32.7 | 34.1 |
| Number of Crowns | 4.8 | 6.9 | 6.0 |
| Leaf Characteristics | | | |
| Terminal Leaflet Length | 8.0 | 6.9 | 8.8 |
| (cm) | | | |
| Terminal Leaflet Width (cm) | 7.0 | 7.2 | 8.3 |
| Terminal Leaflet | 1.14 | 0.96 | 1.06 |
| Length/Width Ratio | | | |
| # Teeth/Terminal Leaflet | 19.9 | 26.0 | 25.7 |
| Color of upper side | 147A | 137A | 137B |
| | dark green | light green | light green |
| Color of under side | 137B | 139C | 139C |
| | light gray | light gray | light gray |
| | green | green | green |
| Petiole Length (cm) | 11.6 | 11.4 | 11.9 |
| Petiole Color | 145A | 141D | 144B |
| | yellow-green | yellow-green | yellow-green |
| Bract Frequency | 90% | 100% | 80% |
| | mostly | | mostly |
| | paired | | paired |
| Stipule Length (cm) | 3.6 | 3.6 | 3.3 |
| Stipule Width (cm) | 2.6 | 2.0 | 2.1 |
| Flower Characteristics | | | |
| Petal Length (cm) | 1.55 | 1.29 | 1.47 |
| Petal Width (cm) | 1.36 | 1.45 | 1.66 |
| Petal Length/Width Ratio | 1.14 | 0.89 | 0.89 |
| Flower Diameter (cm) | 3.6 | 3.36 | 4.14 |
| Calyx Diameter (cm) | 3.08 | 4.17 | 4.82 |
| Flower Color | 155C | 155C | 155C |
| | white | white | white |
| Fruit Characteristics | | | |
| Fruit Length (cm) | 4.9 | 4.1 | 4.6 |
| Fruit Width (cm) | 4.3 | 3.9 | 4.0 |
| Fruit Length/Width Ratio | 1.15 | 1.06 | 1.13 |
| Average Berry Weight (g) | 19 | 18 | 26 |
| External Color | 46A | 46B | 42A |
| | dark red | red | orange red |
| Internal Color | 43A | 44A | 42C |
| | medium red | medium red | light red |
| Achene Coloration | 12A to 178B | 13B to 46A | 12A to 46A |
| | yellow to | yellow to red | yellow to red |
| | dark red | | |
| Yield (g/plant) | 432 | 335 | 539 |

TABLE 2
CHARACTERISTICS OF MADEIRA, BISCAYNE AND MARATHON

| | Madeira | Biscayne | Marathon |
|---|---|---|---|
| Plant | | | |
| Habit | flat globose | flat globose | flat globose |
| Density | medium | medium | medium |
| Vigor | strong | strong | strong |
| Leaf | | | |
| Shape in cross section | concave | concave | concave |
| Interveinal blistering | medium | weak | medium |
| Glossiness | medium | medium | medium |
| Number of leaflets | three only | three only | three only |
| Terminal leaflet | revolute | revolute | revolute |
| margin profile | | | |
| Terminal leaflet shape | obtuse | rounded | slightly |
| of base | | | oblique |
| Terminal leaflet shape | rounded | rounded | obtuse |
| of teeth | | | |
| Stipule pubescence | medium | medium | medium |
| Petiole pubescence | medium | medium | medium |
| Petiole pose of hairs | downwards | outwards | outwards |
| Stolon | | | |
| Number | many | many | medium to |
| | | | many |
| Anthocyanin | strong to | strong | medium to |
| coloration | very strong | | strong |
| Thickness | medium to | medium | medium to |
| | thick | | thick |
| Pubescence | medium | medium | sparse |
| Inflorescence | | | |
| Position relative to | beneath | level | beneath |
| foliage | | | |
| Diameter of calyx | larger | larger | larger |
| relative to corolla on | | | |
| secondary flowers | | | |
| Diameter of inner | same size | same size | same size |
| calyx relative to outer | | | |
| on secondary flowers | | | |
| Spacing of petals | overlapping | overlapping | overlapping |
| Fruiting Truss | | | |
| Fruiting Truss Length | 13.7 | 17.1 | 18.3 |
| (cm) | | | |
| Attitude at first | prostrate | prostrate | prostrate |
| picking | | | |
| Fruit | | | |
| Predominant shape | conical | conical | cordate |
| Difference in shapes | slight | slight | slight |
| between primary and | | | |
| secondary fruits | | | |
| Band without achenes | narrow | narrow | narrow |
| Unevenness of surface | weak | weak | weak |
| Evenness of color | even | even | slightly |
| | | | uneven |
| Glossiness | strong | strong | strong |
| Insertion of achenes | level with | level with | below surface |
| | surface | surface | |
| Insertion of calyx | level | level | level |
| Pose of the calyx | reflexed | spreading | spreading |
| segments | | | |
| Size of calyx in | smaller | larger | larger |
| relation to fruit on | | | |
| secondary fruit | | | |
| Adherence of calyx | strong | strong | strong |
| Firmness of flesh | medium | firm | medium |
| Evenness of flesh | slightly | slightly | slightly |
| color | uneven | uneven | uneven |
| Distribution of flesh | marginal and | marginal and | marginal and |
| color | central | central | central |
| Hollow center size | medium | large | medium |
| Sweetness | weak to | strong | weak to |
| | medium | | medium |
| Texture when tasted | fine | fine | fine |
| Acidity | weak to | medium | medium |
| | medium | | |
| Time of Flowering | very early | early | very early |
| Harvest Interval | Late- | Early- | Early- |
| | November | December | December |
| | through | through | through |
| | Mid-April | Mid-April | Mid-April |
| Type of Bearing | partially | partially | partially |
| | everbearing | everbearing | everbearing |

### Pest and Disease Resistance and Susceptibility

TABLE 3

| | Madeira | Biscayne | Marathon |
|---|---|---|---|
| Reaction to Pests | | | |
| Tetranychus urticae | susceptible | susceptible | susceptible |
| Aphis spp. | susceptible | susceptible | susceptible |
| Lygus hesperus | susceptible | susceptible | susceptible |
| Reaction To Diseases | | | |
| Botrytis fruit rot | moderately | moderately | moderately |
| | susceptible | susceptible | susceptible |
| Powdery mildew | moderately | susceptible | susceptible |
| | susceptible | | |
| Verticillium wilt | moderately | susceptible | susceptible |
| | resistant | | |
| Strawberry Mottle Virus | moderately | moderately | moderately |
| | resistant | resistant | resistant |
| Xanthomonas fragariae | moderately | moderately | moderately |
| | susceptible | susceptible | susceptible |

### Isozyme Analysis

In addition to the morphological description above, the new cultivar, Madeira, has been analyzed to obtain an indication of its genetic makeup to provide further means for identifying the new variety and distinguishing it from some other somewhat similar and/or related strawberry varieties. Specifically, leaf samples of Madeira, Biscayne, and Marathon were analyzed by electrophoresis for isozyme patterns of the enzymes phosphoglucoisomerase (“PGI”), leucine aminopeptidase (“LAP”) and phosphoglucomutase (“PGM”). See J. Amer. Soc. Hort. Sci. 106:684-687. Isozyme characterization of the three varieties is presented in Table 4, with the letters representing the banding patterns for each enzyme as designated in the above-identified article.

TABLE 4
ISOZYME ANALYSIS FOR MADEIRA, BISCAYNE AND
MARATHON

| Locus | Madeira | Biscayne | Marathon |
|---|---|---|---|
| PGI | A1 | A1 | A1 |
| LAP | B1 | B3 | B3 |
| PGM | C2 | C4 | C4 |

What is claimed is:

1. A new and distinct variety of strawberry plant, substantially as shown and described.
